(19) United States
(12) Patent Application Publication
(10) Pub. No.: US 2011/0071409 A1
(43) Pub. Date: Mar. 24, 2011

(54) BLOOD PRESSURE THAT DETECTS VASCULAR SCLEROSIS

(75) Inventors: WEI-CHIH HU, CHUNG LI (TW); LIANG-YU SHYU, CHUNG LI (TW); YUAN-TA SHIH, CHUNG LI (TW); YI-JUNG SUN, CHUNG LI (TW); CHEN-HUAN CHEN, TAIPEI (TW); HAO-MIN CHENG, TAIPEI CITY (TW)

(73) Assignee: CHUNG YUAN CHRISTIAN UNIVERSITY, Chung Li (TW)

(21) Appl. No.: 12/716,603

(22) Filed: Mar. 3, 2010

(30) Foreign Application Priority Data
Sep. 24, 2009 (TW) 098132207

(51) Int. Cl. A61B 5/022

(52) U.S. Cl. 600/490

(57) ABSTRACT

A blood pressure monitor that detects vascular sclerosis is revealed. The blood pressure monitor includes a cuff, an air pump, an air escape valve, a pressure sensor, a processing circuit, a first conversion circuit, and an arithmetic circuit. The cuff is arranged at a user's hand and is pumped up and inflated by the air pump while the air escape valve is used to release air from the cuff. The pressure sensor is disposed on the cuff to detect a pressure of the cuff and generate a pressure sensing signal. The processing circuit processes the pressure sensing signal and generates a processed signal that is converted by the first conversion circuit, According to the converted processed signal, the arithmetic circuit calculates a systolic pressure, a diastolic pressure of a user and obtains a vasodilation constant so as to check vascular sclerosis of the user.

### BACKGROUND OF THE INVENTION

1. Fields of the Invention

The present invention relates to a blood pressure monitor, especially to a blood pressure monitor that detects blood vessel hardening (vascular sclerosis).

2. Descriptions of Related Art

Due to lives under high pressure and delicate foods, high blood pressure has always been one of the ten leading causes of death. Besides control of foods, people have to monitor their blood pressure for prevention of high blood pressure. In recent years, cardiovascular disease has also been one of the ten leading causes of death and has being with an increasing rate according to statistics of the department of health. The cardiovascular disease refers to arterial disease (atherosclerosis) so that a hardening of a blood vessel (vascular sclerosis) is one of important indicators of cardiovascular diseases. Once the hardening of blood vessels is discovered early, the cardiovascular disease can be prevented. Thus people got to monitor their blood pressure and the degree of blood vessel hardening so as to check their health conditions. Therefore, both high blood pressure and cardiovascular diseases can be prevented.

Along with increasing incomes, change of population structure, adoption of new medical technology, and some other factors, people have paid more attentions to health and medical and health devices such as blood pressure monitors, glucosemeters, etc., have been essentials for families. Thus it is convenient for users to measure their blood pressure and blood glucose so as to learn their health conditions for disease prevention. Although the medical technology is quite advanced now, there is still no easy way to measure the degree of blood vessel hardening, or an index of vascular stiffness. Thus there is no good measure of vascular stiffness assessment of health conditions. Therefore, cardiovascular disease remains one of the ten leading causes of death.

A conventional way of diagnosis is an intrusion-detection way. The procedures are not only complicated but also time-consuming. Thus the most common index of arterial stiffness adopted now is Pulse Wave Velocity (PWV). It measures the velocity of the blood pressure waveform between two sites and requires two sets of cuffs for measuring blood pressure as well as a single-lead ECG provides a time reference. The two sets of cuffs are arranged at the hand and the ankle respectively so as to obtain the time difference between the pulses of the two sites. Then by the distance between the two sites, the Pulse Wave Velocity is obtained. Generally, the normal PWV is less than 1200 mm/sec. The above way of measurement needs to measure many physiological parameters and the procedures are complicated. Moreover, the medical device with at least two sets of cuffs and one set of ECG signal for measuring the degree of vascular stiffening is not so prevalent. Therefore, people are unable to monitor conditions and changes of the blood vessels for prevention of vascular diseases.

There is a need to develop a blood pressure monitor that also measures vascular sclerosis. The device not only overcomes the above shortcomings but also measure a vasodilation (vascular dilation) constant during measurement of blood pressure by only one set of cuff. The vasodilation constant is used as an indicator that checks the degree of blood vessel hardening so as to solve above problems.

### SUMMARY OF THE INVENTION

Therefore it is a primary object of the present invention to provide a blood pressure monitor that detects vascular sclerosis. According to the pressure of a cuff, a systolic pressure and a diastolic pressure are calculated so as to get a vasodilation constant. While measuring the blood pressure, the vasodilation constant is also obtained. Thus the vascular sclerosis is detected according to the vasodilation constant, Therefore, the detection is simplified and is becoming more prevalent.

It is another object of the present invention to provide a blood pressure monitor that detects vascular sclerosis having a simple structure and getting a vasodilation constant while measuring the blood pressure so as to detect vascular sclerosis and make the detection become more prevalent.

In order to achieve above objects, the present invention provides a blood pressure monitor that detects vascular sclerosis. The blood pressure monitor includes a cuff, an air pump, an air escape valve, a pressure sensor, a processing circuit, a first conversion circuit, and an arithmetic circuit. The cuff is placed around a user's hand and is connected with the air pump to be pumped up for inflation while the air escape valve is for releasing air from the cuff. The pressure sensor is arranged at the cuff to detect a pressure of the cuff and generate a pressure sensing signal. The processing circuit coupled with the pressure sensor processes the pressure sensing signal from the pressure sensor and generates a processed signal. The first conversion circuit coupled with the processing circuit converts the processed signal. According to the converted processed signal, the arithmetic circuit calculates a systolic pressure and a diastolic pressure of a user for getting a vasodilation constant that is used for checking vascular sclerosis of the user.

Moreover, the processing circuit of the blood pressure monitor that detects vascular sclerosis includes an instrumentation amplifier and a filter. The instrumentation amplifier amplifies the pressure sensing signal generated by the pressure sensor while the filter is coupled with the instrumentation amplifier for filtering the pressure sensing signal amplified by the instrumentation amplifier. Then the processed pressure sensing signal is sent to the first conversion circuit for conversion.

Furthermore, a blood pressure monitor of the present invention further includes a second conversion circuit that is coupled with the arithmetic circuit and is able to receive, convert both an inflation control signal and a deflation control signal from the arithmetic circuit, and send the signals to the air pump and the air escape valve respectively for control of the air pump and the air escape valve to inflate and deflate the cuff.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a block diagram of an embodiment of a blood pressure monitor that detects vascular sclerosis according to the present invention;

FIG. 2 is a block diagram of another embodiment of a blood pressure monitor that detects vascular sclerosis according to the present invention.

### DETAILED DESCRIPTION OF THE PREFERRED EMBODIMENT

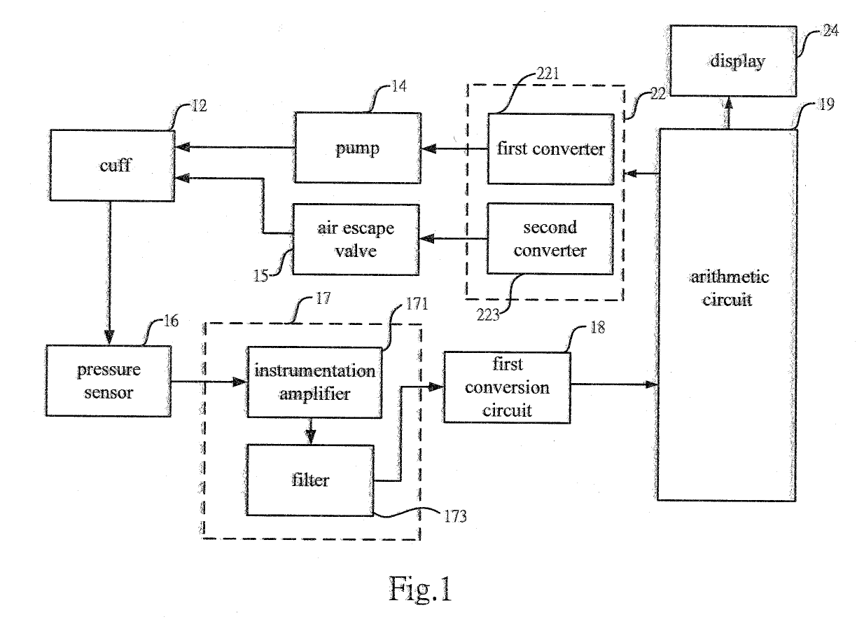

Refer to FIG. 1, a blood pressure monitor that also measures vascular sclerosis includes a cuff 12, an air pump 14, an air escape valve 15, a pressure sensor 16, a processing circuit 17, a first conversion circuit 18, an arithmetic circuit 19, a second conversion circuit 22 and a display 24. The cuff 12 is wrapped around people's hands and is pumped up and inflated by the air pump 14 connected therewith. In this embodiment, the air pump 14 is an electric air pump that inflates the cuff 12 in a linear way. The air escape valve 15 is coupled with the air pump 14 so as to release air in the cuff 12. In this embodiment, the air escape valve 15 is an electric valve or a linear valve that releases air from the cuff 12 in a linear way.

As shown in FIG. 1, the pressure sensor 16 is arranged at the cuff 12 for detecting a pressure of the cuff 12 and generating a pressure sensing signal that is a waveform signal. The processing circuit 17 is coupled with the pressure sensor 16 to process the pressure sensing signal and generate a processed signal which is also a waveform signal. The processing circuit 17 mainly deals with the pressure sensing signal such as amplifying the waveform signals and filtering noises of the waveform signals for convenience of following processes such as conversion and calculation of the first conversion circuit 18 and the arithmetic circuit 19 so as to increase the accuracy. In an embodiment of the present invention, the processing circuit 17 is an analog processing circuit.

The processing circuit 17 of this embodiment includes an instrumentation amplifier 171 and a filter 173. The instrumentation amplifier 171 is coupled with the pressure sensor 16 to amplify the pressure sensing signal while the filter 173 coupled with the instrumentation amplifier 171 is for filtering the amplified pressure sensing signal. If the noise-to-signal ratio is not high, the pressure sensing signal generated from the pressure sensor 16 is amplified by the instrumentation amplifier 171 and then is directly sent to the first conversion circuit 18, without disposition of the filter 173. The above embodiment is only a preferred embodiment of the present invention. The design of the instrumentation amplifier 171 varies according to different kinds of pressure sensors 16, the state of the pressure sensing signal or requirements of the arithmetic circuit 19.

Still refer to FIG. 1, the first conversion circuit 18 connected with the processing circuit 17 is for conversion of the processed signal from an analog signal to a digital signal. In an embodiment of the present invention, the first conversion circuit 18 is an analog-to-digital converter that samples waveform of the processed signal and outputs the sampled result which is a digital signal. The arithmetic circuit 19 coupled with the first conversion circuit 18 is to receive the processed signal being converted by the first conversion circuit 18 and then calculate a systolic pressure, a diastolic pressure and a vasodilation constant of the user according to the received processed signal that represents a pressure change of the cuff 12. The systolic pressure and the diastolic pressure are used as indicators for checking blood pressure while the vasodilation constant is used to check whether the vascular sclerosis happens.

Moreover, the arithmetic circuit 19 is coupled with the display 24 so as to send the measured data of the systolic pressure, the diastolic pressure and the vasodilation constant to the display 24 for users to read. Furthermore, according to the received processed signal, the arithmetic circuit 19 obtains and sends an average blood pressure and a pulse rate to the display 24 for display. In this embodiment, the display 24 is a liquid crystal display (LCD).

In addition, the arithmetic circuit 19 generates an inflation control signal and a deflation control signal for control of the air pump 14 and the air escape valve 15 respectively. The arithmetic circuit 19 in this embodiment is a microprocessor. Once the air pump 14 and the air escape valve 15 can only receive analog signals, the second conversion circuit 22 of the present invention can convert both the inflation control signal and the deflation control signal generated from the arithmetic circuit 19 into analog signals, respectively sent to the air pump 14 and the air escape valve 15. Thus the air pump 14 is controlled to inflate the cuff 12 and the air escape valve 15 is controlled to release air from the cuff 12.

The second conversion circuit 22 is composed of a first converter 221 and a second converter 223. In a preferred embodiment, the first converter 221 as well as the second converter 223 is a digital to analog converter. The first converter 221 is coupled between the arithmetic circuit 19 and the air pump 14 and is used for converting the inflation control signal generated by the arithmetic circuit 19 into an analog signal and sending the analog signal to the air pump 14 so as to control the air pump 14 for inflation of the cuff 12. The second converter 223 coupled between the arithmetic circuit 19 and the air escape valve 15 is for converting the deflation control signal generated by the arithmetic circuit 19 into an analog signal and sending the analog signal to the air escape valve 15 so as to control the air escape valve 15 for air releasing of the cuff 12.

How the systolic pressure, the diastolic pressure and the vasodilation constant are obtained through calculation of the arithmetic circuit 19 is described in following details. In the beginning, the arithmetic circuit 19 generates and sends an inflation control signal to the air pump 14 to inflate the cuff 12. Under the control of the arithmetic circuit 19, the air pump 14 inflates and the inflation is in a linear relationship. The pressure sensor 16 detects the pressure of the cuff 12 and generates a pressure sensing signal correspondingly. The pressure sensing signal is a waveform signal whose waveform oscillates along with the pulse beat. The pressure sensing signal is passing through the processing circuit 17 and the first conversion circuit 18 and then sent to the arithmetic circuit 19. That means the pressure sensing signal is processed by the processing circuit 17 and is converted into an analog signal by the first conversion circuit 18. Thus the signals received by the arithmetic circuit 19 are waveforms that show gradually increasing changes of the pressure detected by the pressure sensor 16. Once the arithmetic circuit 19 checks that the pressure of the cuff 12 has achieved a preset value according to the pressure detected by the pressure sensor 16, the arithmetic circuit 19 controls the air pump 14 stopping the inflation.

Next the arithmetic circuit 19 generates and sends a deflation control signal to the air escape valve 15 so as to control the air escape valve 15 for releasing air from the cuff 12. Thus the pressure inside the cuff 12 is reduced gradually. The air releasing of the air escape valve 15 controlled by the arithmetic circuit 19 is also in a linear relationship. According to waveform of the processed signal, the arithmetic circuit 19 obtains a pulse interval. The releasing rate of the air escape valve 15 controlled by the arithmetic circuit 19 can be adjusted according to the pulse interval. Once the pulse interval is long, the releasing rate is slowed and if the pulse interval is short, the releasing rate is high.

The pressure sensor 16 detects the pressure of the cuff 12 that is reduced gradually and generates a pressure sensing signal correspondingly. The pressure sensing signal generated from the pressure sensor 16 is passing through the processing circuit 17 and the first conversion circuit 18 and then sent to the arithmetic circuit 19. The signals received by the arithmetic circuit 19 are waveforms of the gradually decreasing of the pressure detected by the pressure sensor 16 and the waveforms are changed due to pulse beat. The arithmetic circuit 19 records the received processed signals and calculates the systolic pressure, the diastolic pressure, average blood pressure and the pulse rate of the user according to the received processed signals. The average blood pressure calculated by the arithmetic circuit 19 is determined by a pressure value of a point on the oscillating waveform that reaches a maximum amplitude. And the systolic pressure is defined as a pressure of a point on the waveform reaching about 50% maximum amplitude appeared before the waveform arrives the maximum amplitude while the diastolic pressure is defined by a point having about 50% maximum amplitude on the waveform after the waveform arrives the maximum amplitude. The above mentioned embodiment is only one of the embodiments of the present invention and the calculation way is not limited to the above one. The arithmetic circuit 19 can also obtain the average blood pressure, the systolic pressure, the diastolic pressure by other ways.

According to the vasodilation constant calculated by the arithmetic circuit 19, whether the blood vessels are becoming less elastic is determined. The vasodilation constant that represents an attenuation constant of the waveform signal obtained during deflation of the cuff 12 is given by the following equation:

p=p₀e^{-(αt)}

wherein P is a pressure value corresponding to descending waveform of the waveform signal; P₀ is an initial pressure corresponding to a starting of the descending waveform; e is a constant; a is an attenuation constant (that's the vasodilation constant); t is descending time of a waveform of the waveform signals. In the above equation, the P₀ can be a diastolic pressure. That's the pressure corresponding to the waveform of the diastolic pressure not descending from the peak, also the initial pressure. P is the pressure corresponding to the waveform of the diastolic pressure descending from high level. The P, P₀ and t are values measured. Thus according to above equation, the attenuation constant α is calculated and obtained. Therefore, the arithmetic circuit 19 of the present invention can get the vasodilation constant according to the systolic pressure.

The vasodilation constant is proportional to the PWV so that the PWV of the user is learned by the vasodilation constant. Thus whether user's blood vessels are normal or not can be checked. The calculation of the vasodilation constant mentioned above is by each descending wave of each oscillation waveform during the deflation of the cuff 12 and regression analysis. The arithmetic circuit 19 of the present invention gets the pulse rate by the numbers of the waveforms of the processed signal received.

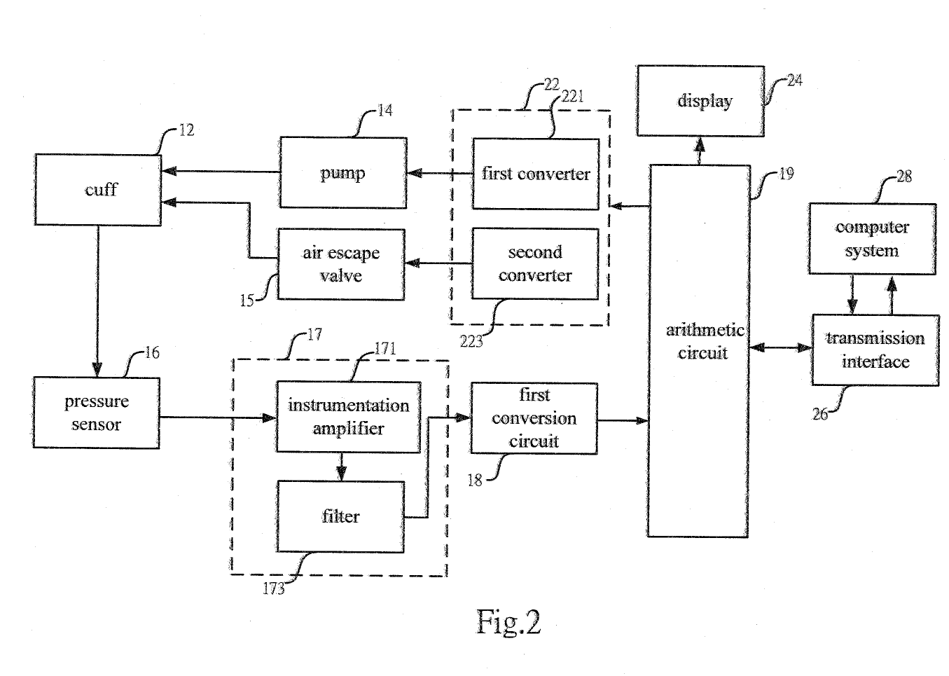

Refer to FIG. 2, a block diagram of another embodiment is revealed. The difference between this embodiment and the above one is in that this embodiment further includes a transmission interface 26 and a computer system 28. The transmission interface 26 is connected with the arithmetic circuit 19 for sending the processed signal converted by the first conversion circuit 18 while the computer system 28 is coupled with the transmission interface 26 for receiving, processing and analyzing the processed signal from the arithmetic circuit 19. For example, the waveform of the pressure sensing signal generated from the pressure sensor 16 is shown on a display of the computer system 28 for further analysis that is carried out for other measurement requirements. In a preferred embodiment of the present invention, the transmission interface 26 is a Universal Serial Bus (USB) or other interface with general specifications.

In summary, a blood pressure monitor that detects vascular sclerosis of the present invention includes a cuff, an air pump, an air escape valve, a pressure sensor, a processing circuit, and an arithmetic circuit. The cuff is arranged at user's hand while the air pump and the air escape valve respectively are used to pump up and release air from the cuff. The pressure sensor detects pressure of the cuff and generates a pressure sensing signal. As to the processing circuit, it processes the pressure sensing signal and generates a processed signal. A first conversion circuit converts the processed signal from the processing circuit. The arithmetic circuit calculates a systolic pressure and a diastolic pressure according to the converted processed signal and obtain a vasodilation constant. Thus the vascular sclerosis is checked according to the vasodilation constant. The blood pressure monitor with simple structure becomes more prevalent. Therefore people can monitor conditions and changes of their blood vessels whenever they want so as to prevent vascular diseases effectively.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A blood pressure monitor that detects vascular sclerosis comprising:
a cuff disposed on a human hand,
an air pump for inflation of the cuff and connected with the cuff,
an air escape valve connected with the air pump and used for releasing air from the cuff,
a pressure sensor arranged at the cuff to detect a pressure of the cuff and generate a pressure sensing signal,
a processing circuit coupled with the pressure sensor, processing the pressure sensing signal and generating a processed signal,
a first conversion circuit coupled with the processing circuit and converting the processed signal, and
an arithmetic circuit coupled with the first conversion circuit and calculating a systolic pressure and a diastolic pressure for getting a vasodilation constant so as to detect vascular sclerosis of the human according to the vasodilation constant.

2. The device as claimed in claim 1, wherein the arithmetic circuit gets the vasodilation constant according to the systolic pressure.

3. The device as claimed in claim 1, wherein the processing circuit includes:
an instrumentation amplifier that amplifies the pressure sensing signal, and
a filter coupled with the instrumentation amplifier and filtering the pressure sensing signal amplified by the instrumentation amplifier to generate the processed signal.

4. The device as claimed in claim 1, wherein the processing circuit is an analog processing circuit.

5. The device as claimed in claim 1, wherein the air escape valve is an electric air escape valve.

6. The device as claimed in claim 1, wherein the air escape valve is a linear escape valve.

7. The device as claimed in claim 1, wherein the air pump is an electric air pump.

8. The device as claimed in claim 1, wherein the first conversion circuit is an analog-to-digital converter that converts the processed signal to a digital signal.

9. The device as claimed in claim 1, wherein the arithmetic circuit is a microprocessor.

10. The device as claimed in claim 1, wherein the arithmetic circuit calculates an average blood pressure and a pulse rate of the human according to the converted processed signal.

11. The device as claimed in claim 1, wherein the blood pressure monitor further includes:
a transmission interface coupled with the arithmetic circuit and sending the processed signal; and
a computer system coupled with the transmission interface and receiving the processed signal to process and analyze the processed signal.

12. The device as claimed in claim 11, wherein the transmission interface is a universal serial bus (USB).

13. The device as claimed in claim 1, wherein the blood pressure monitor further includes:
a display coupled with the arithmetic circuit and used for receiving and displaying the vasodilation constant, the systolic pressure and the diastolic pressure.

14. The device as claimed in claim 13, wherein the display is a liquid crystal display (LCD).

15. The device as claimed in claim 1, wherein the blood pressure monitor further includes:
a second conversion circuit that is coupled with the arithmetic circuit for receiving an inflation control signal and a deflation control signal from the arithmetic circuit and then converts and sends the inflation control signal and the deflation control signal to the air pump and the air escape valve respectively for control of the air pump and the air escape valve.

16. The device as claimed in claim 15, wherein the second conversion circuit includes:
a first converter coupled between the arithmetic circuit and the air pump and used for converting and sending the inflation control signal from the arithmetic circuit to the air pump; and
a second converter coupled between the arithmetic circuit and the air escape valve and used for converting and sending the deflation control signal from the arithmetic circuit to the air escape valve.

17. The device as claimed in claim 16, wherein the first converter and the second converter are both digital to analog converters, respectively converting the inflation control signal and the deflation control signal to analog signals.
